(19) United States
(12) Patent Application Publication
(10) Pub. No.: US 2008/0085210 A1
(43) Pub. Date: Apr. 10, 2008

(54) Decontamination of filtration media for respiration

(76) Inventors: Henry Griesbach, Clarkston, GA (US); Kelly Arehart, Roswell, GA (US); David Wayne Johnson, Alpharetta, GA (US); Gavin MacDonald, Decatur, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/544,064

(22) Filed: Oct. 5, 2006

(51) Int. Cl. A61L 2/00

(52) U.S. Cl. 422/21; 128/206.19

(57) ABSTRACT

Microwave energy may be used to disinfect used face masks, respirators and other items containing filtration media for respiration. A method of disinfecting is provided as well as filtration media having indicators to visually indicate when disinfection has occurred. The indicators may function based on temperature and steam generation during microwaving. Thermochromic materials may be used to indicate that a proper temperature has been reached.

### BACKGROUND OF THE INVENTION

Items containing filtration media for respiration, e.g. face masks and respirators, are typically of limited used and disposed of after a single use. In the event of a severe epidemic outbreak, such as SARS or avian flu or the like, the potential exists to rapidly exhaust the supplies of face masks and respirators due to a rapid peak in demand. The United States government, through the Department of Health and Human Services, has recognized this threat to public health and has asked industry for ways to allow the reuse of respirators, primarily the N95 type, and to develop reusable face masks for health care workers and the public to reduce the person-to-person spread of influenza. The N95 respirators are so designated by the National Institute for Occupational Safety and Health (NIOSH) as recommended for use during high risk activities in health care settings. The term “respirator” refers to all NIOSH certified respirators with N95 or better filters and the term “mask” refers to any procedural or other facial mask.

One potential way of overcoming a respirator shortage would be the re-use of such items, if a reliable and simple method to disinfect them were readily available. Any successful, simple method would have to use commonly available items found at home and at health care facilities and would have to provide an indication that the process was finished.

It is clear that a need exists for a method for clearly signaling when conditions have been achieved for disinfecting filtration media for respiration. There is also a need for a face mask or respirator that may be disinfected by common means and that will provide indication that the disinfection conditions have been achieved.

### SUMMARY OF THE INVENTION

In response to the foregoing difficulties encountered by those of skill in the art we have successfully disinfected filtration media for respiration by subjecting such items to microwave energy for a time sufficient to create conditions that favor disinfection. We have also provided a face mask and respirator containing indicators that provide a visual indication that the disinfection has occurred. The indicators may be incorporated into the filtration media by placing them on the surface of the media or including in the materials from which the media is made.

### DETAILED DESCRIPTION OF THE INVENTION

The present invention involves the use of microwave energy to disinfect used face masks, respirators and any other microwavable item containing filtration media for respiration and methods for the detection of the conditions for disinfection. The terms “face mask” and “mask” as used herein include respirators and other items containing filtration media for respiration like air filters for air conditioning and heating systems and automobile cabin air.

One method to disinfect face masks used a commonly available microwave oven, water, and a container, for example a plastic bag, to demonstrate the efficacy and efficiency of disinfecting face masks contaminated with real world infectious levels of Staph. aureus, a common pathogenic bacteria. The plastic bag container enclosed the face mask and water within a volume smaller than the chamber of the microwave oven. The use of a container smaller than the chamber of the microwave oven is not necessary to achieve conditions of disinfection, however, such a container prevented contamination of the interior of the microwave oven from the face mask itself and probably reduced the time needed to achieve the disinfection conditions immediately surrounding the face mask.

Microwave energy is preferred because it can deliver a relatively large amount of energy to a substrate in a relatively short period of time. This will reduce the damage to the face mask as compared to heating in a traditional radiant or convective oven. In fact, the inventors have found that microwaving for more than 30 seconds can produce conditions conducive to disinfection. More particularly the face mask should be microwaved for between 45 seconds and 2 minutes or, still more particularly between 1 and 2 minutes and still more particularly between 1.5 and 2 minutes. It should be noted that the indicators taught herein will function if microwaved for longer than 2 minutes (in fact there is no upper limit), but it has been found that damage to the face mask usually results from such exposures. Microwave ovens are also preferred because they are quite common. Conventional microwave ovens commonly operate at between 600 and 1000 watts.

A microwave oven works by passing microwave energy, usually at a frequency of 2450 MHz (a wavelength of 12.24 cm), through a material placed within the chamber of the microwave oven. Water, fat, and/or sugar molecules in the material absorb energy from the microwaves in a process called dielectric heating. Many molecules (such as those of water) are electric dipoles; they have a positive charge at one end and a negative charge at the other, and therefore rotate as they try to align themselves with the alternating electromagnetic field induced by the microwaves. This molecular movement creates heat as the rotating molecules hit other molecules which creates friction among them and generates the heat. Microwave heating is most efficient on water (liquid), and less so on fats and sugars which have less molecular dipole moments, and much less on ice (frozen water) where the molecules are not as free to rotate.

Various styles and configurations of multi-layered face masks are well known by those skilled in the art and the present invention is not limited to any particular style or configuration of such multi-layered face masks. The components of the face mask must, however, be made from materials that withstand exposure to microwaves. Face masks suitable for the practice of this invention include those taught in U.S. Pat. Nos. 7,077,139, 7,044,131, 6,427,693, 5,883,026, 4,920,960 and 4,635,628, incorporated herein by reference in their entirety.

Common materials for construction of face masks and filters include spunbond and meltblown fibers and fabrics in various arrangements. The term “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface. Laminates of spunbond and meltblown fibers may be made, for example, by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown (abbreviated as “M”) layers or multiple spunbond (abbreviated as “S”) layers in many different configurations and may include other materials like films (abbreviated as “F”) or coform materials (see U.S. Pat. No. 4,100,324 for descriptions of exemplary “coform” materials), e.g. SMMS, SM, SFS, etc.

The face masks herein had visual color change indicators for detecting temperature change as well as an indicator for detecting the phase transition of water-to-steam. These indicators signaled that conditions favoring disinfection were achieved. The invention can achieve the conditions for disinfection and signal their existence in numerous ways. In one embodiment the indicators may be reversible, therefore allowing the face mask to undergo multiple exposures to microwave energy with each exposure indicating when conditions for disinfection are reached. In another embodiment the indicators are irreversible and signal only once when the condition the indicator is sensing is obtained.

Thermochromism is the ability of a substance to change color due to a change in temperature. A visual color change for detecting temperature is essential for practicing the invention. Materials that change color with temperature, i.e. thermochromatic or thermochromic materials, include pigments, dyes, and microencapsules that allow mixing of segregated of specific chemical combinations at certain temperatures. All of these are examples of ways to achieve such detection.

Thermochromic materials include leuco dyes, liquid crystals and various polymerics, e.g. polythiophenes. While leuco dyes are less precise than liquid crystals in terms of their respective response temperature ranges, the lueco dyes allow wider range of colors to be used.

Thermochromism may be reversible or irreversible. Reversible visual temperature indicators can be thermochromatic dyes; irreversible visual temperature indicators can be, for example, combinations of pseudo-stable emulsions of paraffin oil encapsulated in a thickened outer phase that mix when the outer phases melt at specified temperatures and result in a color change, as taught in, for example, U.S. Pat. No. 3,828,612. The '612 patent teaches an indicator for detecting whether foodstuffs and the like have been subjected to conditions that would have resulted in a substantial deterioration or at least in a defective quality of product. The '612 system includes at least one component that is solid at the intended storage temperature and a solvent. If the system is subjected to a high temperature, e.g. above about 30° C., the solid component partially or completely dissolves, resulting in an irreversible visible indication of over-temperature. In short, the segregated components lose their segregation at temperatures above about 30° C.

Another irreversible indicator may be produced by the inclusion of a polythiophene in, by way of examples, polyolefin, polystyrene and other matrices.

The thermochromatic properties of these polymers, notably poly(3-alkylthiophene) in polyethylene and polypropylene were reported by Lucht, Euler and Gregory in Polymer Preprints, 2002, 43(1), 59. Lucht et al. report a two phase model as temperature is raised, first involving the twisting of the main chain units as the side chain lattice melts and then, as a result of the twisting, an increase in the band gap, thus producing a blue shift in optical absorption. Polythiophenes are commercially available from Plextronics Inc. of Pittsburgh, Pa. Since many face masks are made from polyolefins, particularly polyethylene and polypropylene, it may be possible to include polythiophene with the polyolefin prior to extrusion of the fibers from which the face mask is made. In this manner, virtually the entire face mask may become a visual indicator.

The water-to-steam phase change indicator, when used, should also be located where it is visible when the mask is microwaved. The addition of water is not always required because there is typically enough water present in a face mask after use, either due to moisture in the air of the ambient environment or from retained moisture in the mask from exhalation. The use of a water-to-steam phase change indicator that also serves as a reservoir for water is a way to ensure that appropriate amounts of water are present.

The water is useful because it becomes steam which is known to be useful for disinfection. It is not known whether the steam disinfects and kills bacteria on the mask or whether the temperatures caused by the steam disinfect and kill the bacteria on the mask or whether the bacteria are killed directly by the microwave energy or whether combinations of energies from steam, temperature, and microwaves disinfect and kill the bacteria on the mask. Without wishing to be bound by theory, the inventors believed the conditions for disinfection occur due to the absorption of the microwaves from the microwave oven by the water molecules and their direct conversion via molecular motion into heat. When sufficient heat is generated, water is changed into steam. The steam permeates the environment of the face mask while it continues to absorb microwaves and convert them to heat. Eventually the heat is sufficient to raise the temperature of the mask to a level at which disinfection occurs. The water used herein is therefore optional. If used, water should be present in an amount between 0.1 gm and 1 gm per face mask, though more water would not disable the indicators, merely lengthen the response time.

A container, e.g., plastic bag, is useful to reduce the space of the localized ambient environment surrounding the face mask. As is commonly known, containing items with some amount of water in an enclosure smaller than the chamber of the microwave oven, even if only partially sealed, reduces the amount of time needed to raise the temperature of the item when exposed to microwaves generated by the microwave oven. Conditions for disinfection can be achieved without the use of such containers enveloping the mask since, without them, the chamber of the microwave oven serves the function of creating a localized ambient environment/space around the mask. The only restriction needed when using containers to hold the mask is that such containers withstand exposure to microwaves and the conditions of disinfection.

An exemplary way of producing a visual temperature indicator is through the use of leuco dyes. Leuco dyes are weak organic bases that can react with weak organic acids, or color developers, in a proton-donor acceptor reaction to effect a color change in the dye. While the leuco dye/color developer system does respond to temperature shifts with a color change, encapsulating the leuco dye with the color developer and a polar solvent within a particle isolates the components from the surrounding environment and creates a reversible thermochromic system. These encapsulated systems are generally easier to use and have the advantage of reversibility as compared to the free components.

Leuco dyes respond to temperature changes with a visual change in color such that the composition is colored when below the transition temperature of the solvent and clear or colorless when above the transition temperature of the solvent. The leuco dyes, which are weak organic bases, change from colored to colorless (clear) upon heating and normally function over a 5 to 15° C. temperature range. Changing temperature shifts the equilibrium between the colored or protonated form of the dye, where the proton is generally donated by the color developer (e.g. a weak acid) and the unprotonated or colorless form. Examples of leuco dyes include spirolactones such as fluorans or crystal violet lactone, spiropyrans, fulgides, and the like.

Leuco dyes generally complete a visual color change within a 3 to 4° C. temperature range. If the transition temperature of the leuco dye is reported as 30° C., for example, the color change would occur between approximately 28 and 32° C.

Leuco dyes are available in a number of colors such as red, blue, green, yellow, and black and can be tailored by judicious selection of the polymeric precursor chemistries and emulsion polymerization conditions to produce the desired color change. Further, the transition temperatures can be adjusted by choosing the polar solvent such that it correspond to the desired temperature change. As an example, a relatively high temperature black dye can be obtained from Color Change Corporation of Streamwood, Ill., US as product type LD-Powder.

Multiple color transitions can be obtained by combining either two or more thermochromic materials that have different transition temperatures or by including a non-color changing pigment into the mixture. For example, two thermochromic ingredients could be combined, one with a blue to colorless transition over a 23-26° C. range and the other with a red to colorless transition over a 29 to 31° C. range, to produce for example, a purple to red to colorless transition. The second approach would be to combine a non-color changing red pigment with the blue thermochromic ingredient, thereby producing a purple color initially, but after the blue-to-colorless transition, red becomes the final color.

Microencapsulation serves to protect the components of the leuco dye from their surroundings, to ensure proper functioning and to prevent diffusion of the phase change solvent when it is present in liquid form. Microencapsulated leuco dyes are available from Color Change Corp., Chromatic Technologies, Inc. of Colorado Springs, Colo., US (Dynacolor®), Matsui International Company Inc., Gardena, Calif., US (Chromicolor® AQ-lnk, Fast Blue Type 25) and Thermographic Measurement Co. of the United Kingdom. Adjustment of the thickness and thermal conductivity of the microcapsule shell materials results in changing the responsiveness of the leuco dye to the temperature change. The microcapsules are generally 3 to 5 microns in diameter though they can be made smaller than 1 micron. Incorporation of the microencapsulated leuco dyes into polymer resin masterbatches (available from, for example, Matsui International Company Inc. as Chromicolor® PP Concentrate) allows one to add these dyes directly into the substrates used to make the facemasks, thereby including a color change indicator within the entire facemask.

### Experimental Details and Results

### Materials and Initial Preparation

Microwave Energy Source: A Sharp 8 cubic foot (226.5 liter), 800 watt microwave model R-209KK (from Sharp Industries, Mahwah, N.J.) was used.
Masks used in the study: FLUID-SHIELD® face masks (product code 47107) with the metal nose strip shaping element removed (from Kimberly-Clark, Dallas, Tex.).
Temperature indication: Two spots of Chromicolor AQ-lnk, Fast Blue Type 25 thermochromic ink (from Matsui International Corporation, Gardena, Calif.) with a visual temperature change by 33° C. were placed on each end of the mask to visually indicate if a temperature greater than 33° C. was achieved.
A strip of cotton fabric (3 cm×8 cm, 0.5 gm as average of 3 strips 1.528 gm) was incorporated into each mask to rapidly absorb the bacterial suspension.
Water-to-steam transition indication: The presence of water and the subsequent transition to steam was visually detected using blue self stick notes (cellulose; 1.5 inch×2 inch and ˜0.14 gm; Turquoise color from Corporate Express®, a Buhrmann Company, Amsterdam, Netherlands); when wetted each note was discernably darker in shade than when dry. Addition of 0.1 gm of water per blue self stick note with the mask was used to establish a reservoir of water to ensure the presence of moisture for subsequent steam generation. Any suitable material that shows a discernable color change upon being wetted may be used in place of the cellulosic note (i.e., rayon, Nylon). Two amounts of water were intentionally added in amounts of 0.1 gm and 0.2 gm, as follows:
For the 0.1 gm of water experiments one blue self stick note was used with each mask (samples 1).
For the 0.2 gm water experiments two blue self stick notes were used with 0.1 gm water on each with each mask (samples 2).
No water was added to samples 3 and 4.
Contamination with bacteria: The face masks for samples 1, 2 and 3 were inoculated with 0.1 ml of water containing 100,000 colony forming units (cfu's) of Staph. Aureus (QC grade) on the added cotton strip. Samples 4 received no bacteria.

### Procedure for Exposing Masks to Microwaves

In order to expose the masks to microwaves while ensuring a controlled volume in which to generate steam, each mask was inserted individually into a one quart (0.95 liter) plastic bag so that the mask laid flat within. The open end of the bag was folded over so that the edge of the opening was occluded by the bag itself and the mask within. The mask in the folded-over bag was placed on a supporting white paper plate and inserted into the microwave and the microwave turned on. The masks were positioned so that the visual temperature indicators and water-to-steam indicators were visible through each of the bags and through the glass door of the microwave. A new bag and paper plate was used for each mask and the glass platter was not used as a substrate on which to mount the paper plate. The platter was removed to eliminate possible heat retention caused by repeatedly exposing it to successive microwaving.

Microwave treatments of the masks were as follows:

| | Time (in | |
|---|---|---|
| Water via blue self stick notes | Microwave) | |
| 0.1 gm | 30 | sec |
| 0.1 gm | 2 | min |
| 0.2 gm | 30 | sec |
| 0.2 gm | 2 | min |
| Controls | | |
| None | 30 | sec |
| None | 2 | min |

| | |
|---|---|
| None | no microwave |
| | treatment |

The experiments were conducted in duplicate. The masks were then extracted and the microbe level counted as follows:

### Bacterial Inoculum Preparation

A bacterial culture was prepared before testing of the face masks began, using tubes of sterile 5 ml Trypticase Soy Broth (TSB). A lyophilized culture of Staphylococcus aureus (ATCC #6538) was hydrated in a tube of sterile 5 ml TSB and incubated for 24 hours at 35° C. The culture was transferred (diluted) two more times before the testing began using aliquots of 100 ul (microliters) for each transfer into 5 ml TSB. S. aureus (ATCC #6538) is listed as an opportunistic pathogen by US Pharmocopeia volume XXIV, and is commonly used by the cosmetic industry for preservative efficacy testing.

The stock culture count for S. aureus was determined to be 1.3×10⁹ CFU (colony forming units)/ml. Also, three (3) 1:10 serial dilutions of the stock culture in sterile phosphate buffered saline were performed and dilutions held before testing began. Target bacterial concentration for the testing was approximately 1-3×10⁵ CFU/ml.

### Face Mask Inoculation and Bacterial Extraction

Before microwaving, each piece of cotton fabric on each face mask was inoculated with 100 ul from the third 1:10 dilution of the stock culture mentioned above. This bacterial concentration was determined to be 1.8×10⁵ CFU/ml.

After microwave exposure, the section of each face mask inoculated with S. aureus was aseptically cut out and transferred to sterile 250 ml flasks each containing 25 ml of sterile Letheen broth. Flasks were placed on a reciprocating shaker, and contents were shaken for two (2) minutes at 192 cycles per minute.

Serial dilutions [10¹-10³] were performed on each flask to determine the number of viable Staph. bacteria. Trypticase Soy Agar with 5 weight percent sheep blood was used to plate each dilution, using 100 ul of solution. Plates were inverted and incubated 24 hours at 35° C. before counting.

Counting viable bacterial colony forming units (CFU): After 24 hours incubation at 35° C., bacterial colony forming units on each serial dilution plate (10¹, 10², and 10³) were counted by visual observation with the assistance of a hand held counter. The viable Staph bacterial CFU count from each plate was multiplied by its particular dilution (10, 100 or 1,000) to determine the number of viable Staph bacterial CFU/milliliter.

Study Results

| | Organism count | | Moisture | | Temperature | |
|---|---|---|---|---|---|---|
| | (CFU/ml) | | Indicator | | Indicator | |
| | Microwave time: | | | | | |
| SAMPLES | 30 sec | 2 mins | 30 sec | 2 min | 30 sec | 2 min |
| 1a: | 250 | None | 50% | Dry | Pale | White |
| 0.1 gm water + Staph aureus | | Detected | dry | | blue | |
| 1b: | None | None | 50% | Dry | Pale | White |
| 0.1 gm water + Staph aureus | Detected | Detected | dry | | blue | |
| 2a: | 1.3 × 10⁴ | None | <50% | Dry | White | White |
| 0.2 gm water + Staph | | Detected | dry | | | |
| aureus | | | | | | |
| 2b: | None | None | <50% | Dry | White | White |
| 0.2 gm water + Staph | Detected | Detected | dry | | | |
| aureus | | | | | | |
| 3a (as a postive | 250 | None | — | — | Pale | White |
| control): | | Detected | | | blue | |
| no water + Staph aureus | | | | | | |
| 3b (as a positive | 2.0 × 10⁴ | None | — | — | Pale | White |
| control): | | Detected | | | blue | |
| no water + Staph aureus | | | | | | |
| 4a (as a negative | None | None | — | — | Pale | White |
| control): | Detected | Detected | | | blue | |
| no water + no | | | | | | |
| Staph aureus | | | | | | |
| 4b (as a negative | None | None | — | — | Pale | White |
| control): | Detected | Detected | | | blue | |
| no water + no | | | | | | |
| Staph aureus | | | | | | |

### Observation Details During Experiments

Samples 1a, b microwaved at 30 seconds: Each mask specimen with 0.1 gm water; blue self stick notes partially wet (50% dry) and thermochromic spots became pale blue from deep blue. For one of the specimens, the weight of blue self stick note was 0.235 gm (wet) and 0.007 gm (water left on balance)=0.228 gm which was 0.144 gm after microwaving; therefore 0.084 gm of water was converted to vapor.
Samples 1a, b microwaved at 2 minutes: Each mask specimen with 0.1 gm water; blue self stick note more than 75% dry and both blue thermochromic spots had turned white. Clearly, two minutes of microwaving was more effective than 30 seconds in eliminating bacteria.
Samples 2a, b microwaved 30 seconds: Each mask specimen with 0.2 gm water per two blue self stick notes with 0.1 gm water each; the blue self stick notes were less than 50% dry and blue thermochromic dots were white. For one specimen, weight determinations of two blue self stick notes: 0.281 gm (dry), then 0.459 gm (wet) and 0.019 gm (water left on balance)=0.440 gm which went to 0.341 gm after microwaving, thus 0.099 gm of water converted to vapor. Note the two self stick notes after room drying were recorded as 0.281 gm (returned to original dry weight).
Samples 2a, b microwaved 2 minutes: Each mask specimen with 0.2 gm water per two blue self stick notes with 0.1 gm water each; both blue thermochromic dots were white and both blue self stick notes were dry after microwaving. Again, two minutes of microwaving was more effective than 30 seconds in eliminating bacteria.

### Samples 3a, b microwaved 30 seconds: Mask specimens with no water as positive controls; blue thermochromic dots were pale blue in color (observed for both duplicates).

Samples 3a, b microwaved 2 minutes: Mask specimens with no water as positive controls; blue thermochromic dots were white after microwaving. Two minutes of microwaving was more effective in eliminating bacteria than was 30 seconds.

### Samples 4a, b microwaved for 30 seconds: Mask specimens with no water and no bacteria as negative controls (to determine if material alterations had occurred): thermochromic dots were pale blue in color after microwaving.

### Samples 4a, b microwaved for 2 minutes: Mask specimen with no water and no bacteria as negative controls (to determine if material alteration had occurred): thermochromic dots were white after microwaving.

### Determination of Air Flow Changes Due to Microwave Processing

Air flow through testing was performed on the face masks to determine if there was any detrimental effect of microwaving the masks.

The testing was done using an automated filter tester model 8130 from TSI Incorporated, 500 Cardigan Road, Shoreview, Minn. 55126-3996. TSI Automated Filter Testers are the instruments much of the filter industry relies upon to determine the efficiency of their media and filter assemblies. In fact, the United States National Institute for Occupational Safety and Health (NIOSH) uses Model 8130 for certification testing of respirators per NIOSH regulation 42 CFR, part 84. The TSI Automated Filter Testers give fast results and are user-friendly. In use, the operator puts a material to be tested in the filter holder and pushes dual control buttons to close the filter holder. The entire test runs automatically. The percent penetration, flow rate, and pressure drop are measured, displayed, and printed at the end of the test. Test times can be shorter than 10 seconds.

In the tables below are the TSI 8130 test results conducted on two different types of masks with and without exposure to microwave energy. The masks were CLASSICS surgical masks (product code 48201 from Kimberly-Clark, Dallas, Tex.) and the FLUID-SHIELD® face masks (product code 47107 from Kimberly-Clark, Dallas, Tex.) with the metal nose strip shaping elements removed. The masks were un-pleated and tested with the outside facing of the mask body facing the aerosol challenge.

| | | % Pene- | Delta P |
|---|---|---|---|
| Mask Type | Microwave Condition | tration | (mm H₂O) |
| CLASSIC ® | no microwave energy | 37.3 | 4.9 |
| | exposure | | |
| CLASSIC ® | with no microwave | 39.3 | 4.9 |
| | energy exposure | | |
| CLASSIC ® | microwaved 30 sec + 0.1 gm | 39.5 | 4.7 |
| | H2O | | |
| CLASSIC ® | microwaved 30 sec + 0.1 gm | 38.5 | 5.0 |
| | H2O | | |
| CLASSIC ® | microwaved 120 sec + 0.2 gm | 38.1 | 5.0 |
| | H2O | | |
| CLASSIC ® | microwaved 120 sec + 0.2 gm | 38.6 | 4.8 |
| | H2O | | |
| FLUID-SHIELD ® | no microwave energy | 23.7 | 6.6 |
| | exposure | | |
| FLUID-SHIELD ® | no microwave energy | 24.6 | 6.5 |
| | exposure | | |
| FLUID-SHIELD ® | Microwaved 30 sec + 0.1 gm | 25.5 | 6.3 |
| | H2O | | |
| FLUID-SHIELD ® | Microwaved 30 sec + 0.1 gm | 24.4 | 6.9 |
| | H2O | | |
| FLUID-SHIELD ® | Microwaved 120 sec + 0.2 gm | 25.4 | 5.9 |
| | H2O | | |
| FLUID-SHIELD ® | Microwaved 120 sec + 0.2 gm | 25.8 | 5.9 |
| | H2O | | |

The results show no measured change in filtration efficacy for the microwaved versus control masks.

### Determination of Moisture Addition to a Facemask Through Respiration

An experiment was undertaken to determine the amount of moisture added to a facemask through the normal process of wearing the mask, without the addition of water beyond that provided by respiration of the wearer.

A FLUID-SHIELD® face mask (product code 47107 from Kimberly-Clark, Dallas, Tex.) was weighed and then worn for 20 minutes and weighed again after wearing. The initial weight was 2.5411 gm and after wearing without talking for 20 minutes was 2.5418 gm, indicating the addition of 0.007 gm of moisture. If the wearer talked for half of the 20 minute test period the moisture added was found to be 0.1518 gm (2.5413 gm initial weight minus 2.6648 gm final weight).

This experiment shows that there would be sufficient moisture in the facemask when microwaved directly after use.

The results demonstrate that inoculated face masks can be disinfected using a conventional microwave oven for greater than 30 seconds, without damaging the materials in the mask. This has been experimentally shown in particular for a new 800 watt microwave oven, The visual moisture and water-to-steam phase transition indicator and visual temperature indicators provide valuable feed-back to the user that appropriate conditions, e.g. temperature and steam levels, for disinfection have been reached. This is especially important considering the variety of makes and types of microwaves available, but also in cases of lower power microwaves or older microwave units whose power source is wearing out.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A filtration media for respiration comprising a visual indicator that changes color upon exposure to a sufficient amount of microwave energy, for a time of more than 30 seconds.

2. The filtration media of claim 1 wherein said indicator changes color at a temperature above about 30° C.

3. The filtration media of claim 1 wherein said indicator is a thermochromic material.

4. The filtration media of claim 1 further comprising a water-to-steam phase change indicator.

5. The filtration media of claim 4 comprising water in the amount of from 0.1 to 1 gm.

6. The filtration media of claim 2 comprising segregated components that lose their segregation at temperatures above about 30° C.

7. The filtration media of claim 6 comprising a polythiophene and a polyolefin.

8. The filtration media of claim 7 comprising poly(3-alkylthiophene) and polyethylene or polypropylene.

9. The filtration media of claim 1 comprising two or more thermochromic material having different transition temperatures.

10. A method of disinfecting a filtration media for respiration comprising the steps of incorporating at least one thermochromic material into said media and microwaving said media for more than 30 seconds and until said material changes color.

11. The method of claim 10 further comprising the step of placing an indicator for a phase change of water-to-steam upon said media and providing a source of water to said media, prior to microwaving said media, until said phase change indicator changes color.

12. The method of claim 11 further comprising the step of placing said filtration media and water in a container prior to microwaving.

13. The method of claim 12 wherein said container has a volume of about 0.95 liters.

14. A system of providing factors that favor disinfection of a face mask comprising incorporating at least one thermochromic material into face mask, and providing a source of microwaves to which said used face mask is exposed for a period of more than 30 seconds.

15. The system of claim 14 further comprising providing water and a water-to-steam phase change indicator on at least one surface of said face mask prior to exposing said used face mask to said microwaves.

16. The system of claim 15 further comprising providing a container for said mask and water prior to exposing said used face mask to microwaves.

17. The system of claim 16 wherein said container has a volume of about 0.95 liters.

18. The system of claim 14 wherein said thermochromic material is a leuco dye that changes color at a temperature above about 30° C.

19. The system of claim 14 wherein said thermochromic material comprises a polythiophene and a polyolefin.

20. The system of claim 19 comprising poly(3-alkylthiophene) and polyethylene or polypropylene.
